(19) United States
(12) Patent Application Publication
(10) Pub. No.: US 2018/0318188 A1
(43) Pub. Date: Nov. 8, 2018

(54) Foundation with controllable color change

(71) Applicant: SHANGHAI CO-FUN BIOTECH CO., LTD, Shanghai (CN)

(72) Inventor: Ke PU, Shanghai (CN)

(21) Appl. No.: 16/039,172

(22) Filed: Jul. 18, 2018

Related U.S. Application Data
(63) Continuation-in-part of application No. 14/912,164, filed on Feb. 16, 2016, now abandoned.

(30) Foreign Application Priority Data
Jul. 10, 2014 (CN) 201410328144.X

(51) Int. Cl. A61K 8/29; A61Q 1/02; A61K 8/81

(52) U.S. Cl. CPC A61K 8/29; A61Q 1/02; A61K 2800/26; A61K 2800/42; A61K 8/817

(57) ABSTRACT

The present invention discloses a foundation with a controllable color change and a preparation method thereof. The foundation comprises colorants, wherein the colorants comprise a white colorant and a chromatic colorant; the white colorant and the chromatic colorant are treated by different surface treatment methods; the surface treatment methods comprise a silicone-philic treatment method and an ester-philic silicone-phobic treatment method; and the foundation changes from an original light color to a dark color or from an original dark color to a light color after smearing. The foundation has the advantages of a controllable and fast color changing effect, a high wettability, a strong covering ability, varied colors, good stability, safety and harmlessness, simple production process and the like.

### CROSS REFERENCE TO RELATED APPLICATIONS

This application is a Continuation-in-Part of co-pending application Ser. No. 14/912,164 filed on Feb. 16, 2016, which is a U.S. National Stage of International Application No. PCT/CN2015/079027 filed on May 15, 2015, for which priority is claimed under 35 U.S.C. § 120; and this application claims priority of Application No. 201410328144.X filed in China on Jul. 10, 2014 under 35 U.S.C. § 119; the entire contents of all of which are hereby incorporated by reference.

### BACKGROUND OF THE PRESENT INVENTION

### Field of Invention

The present invention relates to a cosmetic as well as a preparation method thereof, and more particularly to a foundation with a controllable color change, as well as a preparation method thereof.

### Description of Related Arts

Foundation cream/liquid is a very important category of cosmetics, is often used for giving the skin instant and attractive color decoration, can also cover blemishes, and is a basis for good makeup effects. Cosmetic manufacturers usually provide foundation with different colors (such as dark skin color and light skin color) based on user skin colors. The appearance color and the color of applied on the skin of a common foundation cream are basically the same.

In order to create novel visual effects and increase using fun for attracting consumers, a new type of color-changeable foundation has been developed in recent years. Color change refers to that: during applying, foundation cream/liquid is subject to a shear force, and a color of the cream is changed from light (such as pale and white) to dark (such as dark skin color and light skin color); or changed from one (such as light yellow) to another (such as deep red). Such foundation not only covers small wrinkles or blemishes like a common foundation, but also has an obvious color changing effect, which is very novel and suitable for creating a market concept as well as attracting the consumers.

Commercially available color-changeable foundation comprises three types as follows.

1) Cellulose Adhesion Technology

On a surface of inorganic pigments (which usually comprises iron oxide yellow, iron oxide red, iron oxide black, etc.) used in cosmetic foundation, ultra-fine titanium dioxide is adhered by cellulose as an adhesion medium. A preparation method thereof is very close to a Chinese folk traditional food: rice glue ball (which takes fillings as a core, and wet glutinous rice flour is adhered on a surface of the core). In use, after being applied by fingers, the titanium dioxide adhered on the surface of the pigment is separated by a shear force, and an original color of the pigment appears, in such a manner that the foundation changes from white (or pale) to skin color, as shown in FIG. 1.

2) Color-Changeable Pearl Powder Technology

Through refraction and scattering effects of the pearl powder on light, the color changing effect is achieved. When using, the pearl powder, which is emulsified and wrapped in the cream, is applied on skin, in such a manner that the pearl powder is exposed to light for displaying color changing.

3) Defects of Using Acid-Base Indicators:

The foundation is prepared with several acid-base indicators according to a principle that when external pH value changes, colors of the acid-base indicators will change. In use, the acid-base indicators contact with the skin (whose pH is slightly acidic), then the color thereof changes, resulting in color changing effect.

In practice, all the above three types of color-changeable foundation have serious defects as follows.

1) Defects of the Cellulose Adhesion Technology:

(1) weak covering ability: wherein a particle size (particle diameter) of the inorganic pigment and a shape thereof are generally stable (for example, particle of iron oxide red is generally spherical with a diameter of 0.3-0.5 μm, and iron oxide black is typically cubic with a diameter of about 0.3 μm); in order to achieve good adhesion wrapping effect, a particle size of the titanium dioxide should be as small as possible (commonly nano titanium dioxide); furthermore, in order to achieve fast and significant color changing effect, an amount of the titanium dioxide adhered on the surface of the inorganic pigment surface should not be too many, and a thickness of an adhesion layer should not be too thick; therefore, it is difficult to achieve sufficient covering ability;

(2) slow color change:

during using the foundation, there is a smearing process; iron oxide particles wrapped by the titanium dioxide are broken under the shear force, in such a manner that the iron oxide particles wrapped are exposed for providing the color changing effect;

therefore, color change speed depends on magnitude of force, speed of smearing, and firmness of adhered particles; wherein slowly and gently smearing is not conducive to breaking the particles, which means not conducive to showing the color changing effect; however, as a cosmetic for face, a foundation cream/liquid is hardly to be rapidly and repeatedly smeared with a larger force during using, which lowers the color changing speed and effect of such foundation;

(3) poor stability:

in a long-term storage process or under extreme storage and transportation conditions, “bleeding phenomenon” is very likely to occur, and the inorganic pigment spontaneously bleeds out and develops, which destroys the appearance and state of the product;

(4) difficulty of controlling production process:

firmness of cellulose adhesion is a contradiction: if the firmness is too high, the foundation cream/liquid is difficult to provide the color changing effect during using; if the firmness is too low, the particles of the foundation cream/liquid will be broken during producing and packaging, which expose the inorganic pigments too early;

therefore, during actual production, the cellulose adhered color-changeable foundation requires a lot during production: paddle must not scrape walls, powder need to be pre-soaked following a strict time and temperature, and emulsifying time and temperature must be strictly controlled; otherwise, product quality is difficult to be controlled.

2) Defects of the Color-Changeable Pearl Powder Technology:

(1) insufficient color changing effect:

before smearing, the color-changeable pearl powder is hidden in the cream and is not exposed to light, so as to hiding color effects of the pearl powder; after smearing, the pearl powder is applied on the skin and exposed to the light, so as to show colors; which is a color changing principle of such technology;

however, the color effect of the pearl powder is much weaker than that of the iron oxide, while main role of the foundation cream is to cover blemishes and adjust skin color, where the pearl powder is inadequate;

(2) shiny effect of pearl powder, which is not suitable for entire face:

the pearl powder naturally has a shiny pearl effect, which is more suitable for eye shadow, lip gloss and other areas in a makeup process; however, the foundation is a make-up basis for the entire face; it will be very unnatural if the entire face is shiny.

3) Defects of using acid-base indicators:

(1) there is only a color changing effect without modifying and protecting effects which are essential for foundation products;

there is only a color changing process, which is not able to cover blemishes and adjust the skin color;

(2) harm to human body without benefit:

commonly used acid-base indicators are mainly: nitrophenol, phenolphthalein, thiosulfate phenolphthalein and azo compounds; which basically belong to organic weak acids or organic weak bases; most of these agents are not on lists of cosmetic raw materials, which means it is not allowed to use such agents in cosmetics.

### SUMMARY OF THE PRESENT INVENTION

Based on the defects of the above existing color-changeable foundation preparation technologies, it is necessary to provide a technology capable of preparing a color-changeable foundation that is safe, harmless, controllable in color changing effect, fast in color change, good in product stability, unlikely to bleed during storage, strong in covering ability after application and rich in color.

The present invention overcomes defects of the above technologies from a technical origin, wherein different surface treatment schemes are adopted for special surface treatment on colorants used in the foundation to artificially enlarge the surface property difference of the colorants of different colors, so that the distribution states thereof in the formula are different, and when applied on the skin, the colorants of different colors are redistributed under a shear force to show another color. A color changing effect of the color-changeable foundation prepared with the scheme of the present invention is controllable and rapid; a covering ability is strong; stability is good; colors are varied; safety and harmlessness are guaranteed; and preparation is simple.

The technical solution specifically adopted by the present invention is:

Colorants are “soul” raw materials of a foundation, particularly for a color-changeable foundation. One foundation comprises colorants of different colors, comprising at least one white colorant, and at least two chromatic colorants, wherein the colors of these colorants need to be shown at different stages. The common white colorants are titanium dioxide, zinc oxide, barium sulfate, etc. The common chromatic colorants are iron oxide yellow, iron oxide red, iron oxide black, chromium oxide green, chromium hydroxide green, ultramarine blue, ultramarine purple, ultramarine pink, etc.

The present invention adopts completely different surface treatment methods for the white colorant and the chromatic colorant to artificially enlarge the surface property difference of the colorants of different colors, so that the distribution states thereof in the formula are different, and when applied on the skin, the colorants of different colors are redistributed under a shear force to show another color. On the basis of obtaining different surface treatment colorants, a specific foundation formula is developed, which is of a water-in-oil type or a water-in-silicone type, that is, the continuous phase is an oil phase and the dispersed phase is a water phase.

The present invention adopts the following two surface treatment methods A and B. When the method A is adopted for the white colorant, the method B is adopted for the chromatic colorant; and when the method B is adopted for the white colorant, the method A is adopted for the chromatic colorant.

(1) Surface Treatment Method A

The surface treatment method A is a silicone-philic treatment method, the treatment agent is organosiloxane, which may be methyl triethoxy silane, dimethicone, dimethiconol, polymethylhydrosiloxane, amodimethicone, aminopropyl polydimethyl siloxane, polypropylsilsesquioxane, polydimethyl siloxane/vinyl polydimethyl siloxane polymer, etc. Preferred is methyl triethoxy silane, dimethicone, dimethiconol, polymethylhydrosiloxane or polydimethyl siloxane/vinyl polydimethyl siloxane polymer.

The adding concentration of the organosiloxane is 0.5% to 15%, preferably 1.5% to 12%, more preferably 2% to 10%, based on the mass of the colorants.

In the organosiloxane treatment, a solvent is selected first to dissolve and dilute the organosiloxane to obtain a surface treatment agent just before using. When dissolving, mixing may be carried out for 10 minutes in a mixer at a revolving speed of 200-300 rpm. The solvent may be low-viscosity (1.5˜10 mm2/s at 25° C.) polydimethyl siloxane, cyclopentasiloxane, cyclohexasiloxane, isopropanol, ethanol, 95% alcohol, etc.

The organosiloxane treatment refers to that: the surface treatment agent prepared is sprayed to the surface of the colorant powder in the form of mist droplets while the colorant powder is mixed at a high speed in a high-speed mixer, and continuous mixing is performed for sufficiently mixing the powder with the surface treatment agent uniformly. The powder is taken out and sent into an oven and dried at certain temperature for a period of time. The drying temperature is 80° C. to 150° C., preferably 90° C. to 130° C., more preferably 100° C. to 120° C. The drying time is 1 to 10 hours, preferably 2 to 8 hours, more preferably 3 to 6 hours.

(2) Surface Treatment Method B

The surface treatment method B is an ester-philic silicone-phobic treatment method, and the treatment agent is a long-carbochain silane coupling agent, a monoalkoxy type titanate coupling agent, a fatty acid or a salt thereof.

The long-carbochain silane coupling agent may be octyl triethoxy silane, hexadecyl triethoxy silane, hexadecyl trimethoxy silane, octadecyl triethoxy silane or octadecyl trimethoxy silane. Preferred is octyl triethoxy silane, hexadecyl triethoxy silane or octadecyl triethoxy silane.

The monoalkoxy type titanate coupling agent may be trioleoyl isopropoxytitanate, tris(dioctylphosphoryloxy)isopropoxytitanate, or isooropyl titanium triisostearate, preferably isooropyl titanium triisostearate.

The adding concentration of the ester-philic silicone-phobic treatment agent is 0.5% to 15%, preferably 1.5% to 12%, more preferably 2% to 10%, based on the mass of the colorants.

For the treatment with the monoalkoxy type titanate coupling agent, a solvent is selected first to dissolve and dilute the monoalkoxy type titanate coupling agent to obtain a surface treatment agent just before using. The mixing ratio of the solvent to the monoalkoxy type titanate coupling agent is 1 to 1 to 10:1, preferably 1.5 to 8:1, more preferably 2 to 5:1. When dissolving, mixing may be carried out for 10 minutes in a mixer at a revolving speed of 200-300 rpm under the heating condition of 80° C. The solvent may be isopropanol, ethanol, 95% alcohol, etc.

The treatment with the monoalkoxy type titanate coupling agent refers to that: the surface treatment agent prepared is sprayed to the surface of the colorant powder in the form of mist droplets while the colorant powder is mixed at a high speed in a high-speed mixer, and continuous mixing is performed for sufficiently mixing the powder with the surface treatment agent uniformly. The powder is taken out and sent into an oven and dried at certain temperature for a period of time. The drying temperature is 80° C. to 150° C., preferably 90° C. to 130° C., more preferably 100° C. to 120° C. The drying time is 1 to 10 hours, preferably 2 to 8 hours, more preferably 3 to 6 hours.

The fatty acid or the salt thereof may be a stearic acid or a salt thereof, an isostearic acid or a salt thereof, a lauric acid or a salt thereof, a myristic acid or a salt thereof, a palmitic acid or a salt thereof. Preferred is the stearic acid or the salt thereof, the isostearic acid or the salt thereof.

The adding concentration of the fatty acid or the salt thereof is 0.5% to 15%, preferably 1.5% to 12%, more preferably 2% to 10%, based on the mass of the colorants.

In the treatment with the fatty acid or the salt thereof, a solvent is selected first to dissolve the fatty acid or the salt thereof at 60° C. to obtain a surface treatment agent just before using. The mixing ratio of the solvent to the fatty acid is 1 to 10:1, preferably 2 to 8:1, more preferably 3 to 5:1. When dissolving, mixing may be carried out for 10 minutes in a mixer at a revolving speed of 200-300 rpm under the heating condition of 80° C. The solvent may be isopropanol, ethanol, etc.

The treatment with the fatty acid or the salt thereof refers to that: the surface treatment agent prepared is sprayed to the surface of the colorant powder in the form of mist droplets while the colorant powder is mixed at a high speed in a high-speed mixer, and continuous mixing is performed for sufficiently mixing the powder with the surface treatment agent uniformly. The powder is taken out and sent into an oven and dried at certain temperature for a period of time. The drying temperature is 80° C. to 150° C., preferably 90° C. to 130° C., more preferably 100° C. to 110° C. The drying time is 1 to 10 hours, preferably 2 to 8 hours, more preferably 3 to 6 hours.

In the foundation of the present invention, adopted is a combination of a white colorant treated by the surface treatment method A and a chromatic colorant treated by the surface treatment method B, or a combination of a white colorant treated by the surface treatment method B and a chromatic colorant treated by the surface treatment method A. If the white colorant and the chromatic colorant are simultaneously treated by the surface treatment method A or the surface treatment method B, a color changing effect cannot be achieved.

In the foundation of the present invention, the percentage of quality of the colorants in the foundation formula added is 1 to 25%, preferably 5 to 20%, more preferably 8 to 15%. In the colorants, the amount of the white colorant is 70 to 98%, preferably 75 to 95%, based on the total amount of the colorants, and the rest is the chromatic colorant.

In the foundation of the present invention, if it is desired to achieve a color changing effect from light or dark during smearing, the white colorant used is subject to the silicone-philic surface treatment, and the chromatic colorant is subject to the ester-philic silicone-phobic surface treatment, that is, the colorants should be combined by the white colorant treated by the surface treatment method A and the chromatic colorant treated by the surface treatment method B. The total amount of the colorants added to the foundation is 2 to 25%, preferably 5 to 20%, more preferably 8 to 15%, based on the total mass of the foundation. The surface treatment method for the white colorant is preferably methyl triethoxy silane, polydimethyl siloxane, polymethylhydrosiloxane, polydimethyl siloxane/vinyl polydimethyl siloxane polymer or polydimethyl siloxane alcohol surface treatment in the surface treatment method A, more preferably, methyl triethoxy silane, polydimethyl siloxane/vinyl polydimethyl siloxane polymer or polydimethyl siloxane alcohol surface treatment. The adding concentration of the treatment agent is 0.5% to 12%, preferably 1.5% to 9%, more preferably 2% to 5%, based on the mass of the white colorant. The amount of the white colorant added to the foundation is 83 to 98%, preferably 85 to 95%, more preferably 87 to 92%, based on the total amount of the colorants. The surface treatment method for the chromatic colorant is preferably octyl triethoxy silane or isooropyl titanium triisostearate surface treatment in the surface treatment method B. The adding concentration of the treatment agent is 0.5% to 12%, preferably 1.5% to 9%, more preferably 2% to 6%, based on the mass of the chromatic colorant. The amount of the chromatic colorant added to the foundation is 2 to 17%, preferably 5 to 15%, more preferably 8 to 13%, based on the total amount of the colorants.

In the foundation of the present invention, if it is desired to achieve a color changing effect from dark or light during smearing, the white colorant used is subject to the ester-philic silicone-phobic surface treatment, and the chromatic colorants are subject to the silicone-philic surface treatment, that is, the colorants should be combined by the white colorant treated by the surface treatment method B and the chromatic colorant treated by the surface treatment method A. The total amount of the colorants added to the foundation is 2 to 25%, preferably 5 to 20%, more preferably 6 to 14%, based on the total mass of the foundation. The surface treatment method for the white colorant is preferably octyl triethoxy silane or isooropyl titanium triisostearate surface treatment in the surface treatment method B. The adding concentration of the treatment agent is 0.5% to 11%, preferably 1% to 8%, more preferably 2% to 5%, based on the mass of the white colorant. The amount of the white colorant added to the foundation is 73 to 95%, preferably 78 to 92%, more preferably 82 to 90%, based on the total amount of the colorants. The surface treatment method for the chromatic colorant is preferably methyl triethoxy silane, polydimethyl siloxane/vinyl polydimethyl siloxane polymer, or polydimethyl siloxane alcohol surface treatment in the surface treatment method A. The adding concentration of the treatment agent is 0.5% to 12%, preferably 1.5% to 8%, more preferably 2% to 6%, based on the mass of the chromatic colorant. The amount of the chromatic colorant added to the foundation is 5 to 27%, preferably 8 to 22%, more preferably 10 to 18%, based on the total amount of the colorants.

The foundation of the present invention is of a water-in-oil type, wherein the oil phase is a continuous phase, and the water phase is a dispersed phase.

In the foundation of the present invention, the oil phase at least comprises a water-in-oil emulsifier, which may be selected from one or more of PEG-10 polydimethyl siloxane, PEG-12 polydimethyl siloxane, PEG-9 polydimethyl siloxyethyl dimethicone, lauryl PEG-9 polydimethyl siloxyethyl dimethicone, cetyl PEG/PPG-10/1 polydimethyl siloxane, lauryl PEG/PPG-18/18 polydimethyl siloxane, PEG-12 polymethyl siloxane/PPG-20 cross-linked polymer and PEG/PPG-18/18 polydimethyl siloxane, added in an amount of 0.1 to 10%, preferably 0.5 to 5%.

In the foundation of the present invention, the oil phase further comprises at least an emollient, and the emollient may be selected from silicone, natural oil, synthetic ester, etc. The silicone may be selected from one or more of dimethicone, cyclopentasiloxane, cyclohexasiloxane, methyl polytrimethyl siloxane, octyl polymethyl siloxane and phenyl trimethicone, added in an amount of 1 to 50%, preferably 3 to 30%, more preferably 5 to 20%; the natural oil may be selected from one or more of coco-caprylate/caprate, C9-12 alkane, castor oil, hydrogenated castor oil, jojoba oil, caprylic/capric triglyceride and the like, added in an amount of 0.1 to 10%, preferably 0.2 to 8%, more preferably 0.5 to 5%; and the synthetic ester may be selected from one or more of propyl heptyl caprylate, dioctyl carbonate, caprylic/capric/myristic/stearic triglyceride, isopropyl myristate, isononyl isononanoate, hydrogenated polydecene, octyldodecanol myristate and hydrogenated polyisobutylene, added in an amount of 0.1 to 10%, preferably 0.5 to 8%, more preferably 1 to 5%.

In the foundation of the present invention, the oil phase further comprises at least a thickening suspension, which may be selected from one or more of disteardimonium hectorite, quaternary ammonium salt-18 bentonite and stearalkonium hectorite, added in an amount of 0.1 to 5%, preferably 0.2 to 3%, more preferably 0.5 to 2.5%.

In the foundation of the present invention, the water phase comprises at least deionized water in an amount of 10 to 60%, preferably 15 to 45%, more preferably 20 to 40%.

In the foundation of the present invention, the water phase further comprises at least a humectant, which may be selected from at least two of glycerin, propylene glycol, butanediol, pentanediol and hexanediol, added in an amount of 1 to 30%, preferably 3 to 25%, more preferably 5 to 20%.

In the foundation of the present invention, the white colorant treated by the surface treatment method A or B and the chromatic colorant treated by the surface treatment method B or A may be directly added into the formula in a certain ratio, or the white colorant is previously mixed with the chromatic colorant into pre-mixed powder in a desired ratio, which is then added to the formula.

If the pre-mixed powder is added to the formula of the foundation, the pre-mixed powder is prepared by: respectively performing surface treatment on the white colorant and the chromatic colorant, and then sufficiently mixing the white colorant (such as at least one of titanium dioxide, zinc oxide and the like) with the chromatic colorant (such as at least two of iron oxide yellow, iron oxide red, iron oxide black, ultramarine blue, chrome oxide green, etc.) in a certain ratio through a high-speed mixer. The following examples can be explained:

Pre-mixed powder for a color-changeable foundation cream/liquid, comprising: titanium dioxide and an inorganic pigment, wherein the titanium dioxide is processed with a methyl triethoxy silanization surface treatment, and this treatment method is the aforementioned surface treatment method A, i.e., silicone-philic treatment; the inorganic pigment is processed with an octyl triethoxy silanization surface treatment, and this method is the aforementioned surface treatment method B, i.e., ester-philic silicone-phobic treatment. According to the present invention, totally different surface treatments are provided to the titanium dioxide and the inorganic pigment (iron oxide) in the foundation cream/liquid, in such a manner that surface properties of such two kinds of powder are quite different. After different surface treatments, the surface properties of the inorganic pigment and the titanium dioxide are quite different as follows.

Octyl groups of eight carbon atoms are connected to an end of a surface silicon atom of the inorganic pigment. Due to being long, a carbochain thereof has a sufficient dispersity in fat of an aliphatic series; while the carbochain has a poor compatibility in oxosilane.

A methyl group of a carbon atom is connected to an end of a surface silicon atom of the titanium dioxide. Due to being short, a carbochain thereof has a sufficient compatibility in the oxosilane.

Therefore, the color-changeable foundation cream/liquid prepared with the pre-mixed powder of the present invention has technical effects such as a controllable and rapid color changing effect, a strong covering ability, varied colors, safety and harmlessness.

According to the pre-mixed powder of the present invention for the color-changeable foundation cream/liquid, the inorganic pigment comprises iron oxide yellow, iron oxide red and iron oxide black. The inorganic pigment may be other inorganic pigments for foundation. By adjusting contents of the inorganic pigment with different colors, varied foundation cream/liquid is able to be prepared.

According to the pre-mixed powder of the present invention for the color-changeable foundation cream/liquid, detail surface treatment methods may be conventional surface treatment methods as long as same technical effects are guaranteed. After the methyl triethoxy silanization surface treatment, a surface of the titanium dioxide is characterized in that a methyl group of a carbon atom is connected to an end of a surface silicon atom of the titanium dioxide; after the octyl triethoxy silanization surface treatment, a surface of the inorganic pigment is characterized in that octyl groups of eight carbon atoms are connected to an end of a surface silicon atom of the inorganic pigment.

Accordingly, the present invention further provides preferred surface treatment methods, in such a manner that a better surface treatment effect is achieved, the surface properties of the titanium dioxide and the inorganic pigment are more stable, and distribution is evener.

A method of the methyl triethoxy silanization surface treatment for the titanium dioxide preferably comprises steps of:

thoroughly mixing 0.45 kg methyl triethoxy silane with 1.05 kg 95% ethanol for obtaining a surface treatment agent just before using;

adding 15 kg titanium dioxide into a high-speed mixer; under a high mixing speed, spraying the surface treatment agent prepared into the high-speed mixer with a spray tank, and thoroughly stirring for thoroughly mixing the surface treatment agent with the titanium dioxide; and

discharging powder and instantly sending into an oven, heating at 105° C. for 4 h, and naturally cooling before discharging.

A reaction process is shown in FIG. 2. After treatment, surface properties of the powder are shown in FIG. 3.

A method of the octyl triethoxy silanization surface treatment for the inorganic pigment preferably comprises steps of:

thoroughly mixing 0.45 kg octyl triethoxy silane with 1.05 kg 95% ethanol for obtaining a surface treatment agent just before using;

adding 15 kg inorganic pigment into a high-speed mixer; under a high mixing speed, spraying the surface treatment agent prepared into the high-speed mixer with a spray tank, and thoroughly stirring for thoroughly mixing the surface treatment agent with the inorganic pigment; and

discharging powder and instantly sending into an oven, heating at 105° C., or 70° C. if the inorganic pigment is the iron oxide yellow, for 4 h, and naturally cooling before discharging.

A reaction process is shown in FIG. 4. After treatment, surface properties of the powder are shown in FIG. 5.

According to the pre-mixed powder for color-changeable foundation cream/liquid provided by the present invention, after the surface treatments, a content of the titanium dioxide is 75-95 wt %, and a rest is the inorganic pigment. For example, a preferred color-changeable pre-mixed powder CC807 comprises (wt %):
the titanium dioxide treated by methyl triethoxy silane: 88%;
the iron oxide yellow treated by octyl triethoxy silane: 7.6%;
the iron oxide red treated by octyl triethoxy silane: 3%;
the iron oxide black treated by octyl triethoxy silane: 1.4%.

According to the present invention, through certain surface treatment methods, differences between the surface properties of the titanium dioxide and the inorganic pigment are artificially increased; then certain formulas are adopted, in such a manner that the titanium dioxide and the inorganic pigment are in different distribution states in an entire emulsification system, so as to control the color changing effects.

Furthermore, the present invention provides a foundation cream/liquid with a controllable color change, comprising the pre-mixed powder for color-changeable foundation cream/liquid. The foundation cream/liquid further comprises an emulsifier, an emollient, a preservative and an fragrance according to requirements. The emollient is a silane emollient and so on. A preparation method of the foundation cream/liquid may be a conventional foundation cream/liquid preparation method.

However, for optimizing the color changing effect of the pre-mixed powder of the present invention, technical requirements for emulsifying the pre-mixed powder in the foundation cream/liquid are strict, which means the pre-mixed powder must be fully emulsified in the foundation cream/liquid, and evenly dispersed.

Therefore, according to the present invention, a formula and a preparation method of a color-changeable foundation cream/liquid which specifically use the pre-mixed powder are further optimized, which enables a best effect of the pre-mixed powder during using.

Preferably, a formula and a preparation method of a color-changeable foundation cream/liquid are as follows.

The formula is:

TABLE 1

| phase | | |
|---|---|---|
| number | component name/provider | content (Wt) % |
| A | Abil EM180 (Degussa) | 0.75 |
| | Abil EM90 (Degussa) | 0.75 |
| | Sensil 5060 silane emollient | 2.20 |
| | (Shanghai Scinecoo) | |
| | DC9040 silicone elastomer (Dow Corning) | 1.00 |
| | DC345 (Dow Corning) | 5.00 |
| | DC556 (Dow Corning) | 1.60 |
| | DC200 (5CST) (Dow Corning) | 5.00 |
| B | color-changeable pre-mixed powder | 8.00 |
| C | deionized water | 100 |
| | 1% ethyloic-β-glucan | 3.00 |
| | (Angel Yeast C90) | |
| | sodium chloride | 1.00 |
| | betaine(trimethylglycine) | 3.00 |
| | 1,3-butanediol | 15.00 |
| D | preservative | appropriate amount |
| | fragrance | appropriate amount |

The preparation method comprises steps of:

1) mixing and stirring an A phase, keeping homogeneity for 1 min, and thoroughly dispersing with a DC9040 silicone elastomer;

2) heating the A phase to 55-60° C., then adding B phase powder, and keeping homogeneity for 1 min for thoroughly dispersing the powder;

3) meanwhile, heating a C phase to about 55-60° C. for completely melting;

4) increasing a stirring speed of the A and B phases (to 200-300 rpm), slowly adding the C phase into the A and B phases for completely emulsifying;

5) cooling by stirring until a temperature is lower than 45° C., then adding a D phase in such a manner that cream becomes white, and discharging; and

6) during cooling, keeping a stirring speed (at 200-300 rpm), wherein the cream becomes white within 2-3 h.

The above formula and preparation method adopt a specific formula and an optimized process. With a specific technique of mixing the A, B, C and D phases, the pre-mixed powder of the present invention is fully emulsified in the foundation cream/liquid, and is evenly dispersed. Furthermore, color is rapidly changed during using, which optimizes an effect.

Accordingly, advantages of the present invention are as follows.

1) Rapid Color Change

After gently applying the color-changeable foundation of the present invention on skin, the color changing effect will instantly show.

2) Strong Covering Ability

According to the color-changeable foundation of the present invention, a particle size thereof is not limited, and an addition amount in the formula is not limited much, while an excellent covering ability is achieved. Strong enough effects of covering skin blemishes and modifying skin color can be provided, while the interestingness of the color-changeable foundation is met.

3) Varied Colors and Adjustable Chroma

According to the color-changeable foundation of the present invention, an addition amount of the titanium dioxide in the formula is not limited much, which enables a very good coloring effect. By adopting a combination of different surface treatment methods A and B, an effect from light to dark can be achieved, an effect from dark to light can also be achieved, and the foundation is more diverse in color and more interesting.

4) Safety

The color-changeable foundation treated by the present invention comprises no acid-base indicator, thereby causing no harm to the skin. The above surface treatment methods have been practiced for many years in cosmetic applications, which are proved to be safe and harmless.

5) Simple Production Technique

The production technique is simple and controllable, and materials used are shear-resistant, extrusion-resistant, and vibration-resistant, which are suitable for transportation by cosmetic manufacturers.

In order to achieve a sufficient color changing effect, the color-changeable cream formula designed by the present invention adopts the emulsion system with silicone oil wrapping water. Although the foundation feels silky, breathable and comfortable, a moisture effect thereof is insufficient. Therefore, in autumns and winters, other skin care products are needed.

6) Good Stability

Through 3 months of 48° C. heat storage, three times of −10 to 48° C. freeze-thaw cycles, preservation at normal temperature for 1 year and other tests, the appearance of the foundation is stable, and no “bleeding phenomenon” occurs, which shows that the stability of the foundation is very good in the long-term storage process or under extreme storage and transportation conditions.

### BRIEF DESCRIPTION OF THE DRAWINGS

FIG. 1 illustrates a principle of a cellulose adhesion technology according to a prior art.

FIG. 2 illustrates a reaction process of surface treatment of a surface treatment method A for titanium dioxide as a white colorant (taking methyl triethoxy silane as an example) according to the present invention.

FIG. 3 illustrates chemical properties of a surface of powder after the surface treatment of the surface treatment method A for titanium dioxide as a white colorant (taking methyl triethoxy silane as an example) according to the present invention.

FIG. 4 illustrates a reaction process of surface treatment of a surface treatment method B for an iron oxide series inorganic pigment as a chromatic colorant (taking triethoxy octyl silane as an example) according to the present invention.

FIG. 5 illustrates chemical properties of a surface of powder after the surface treatment of the surface treatment method B for an iron oxide series inorganic pigment as a chromatic colorant (taking methyl triethoxy silane as an example) according to the present invention.

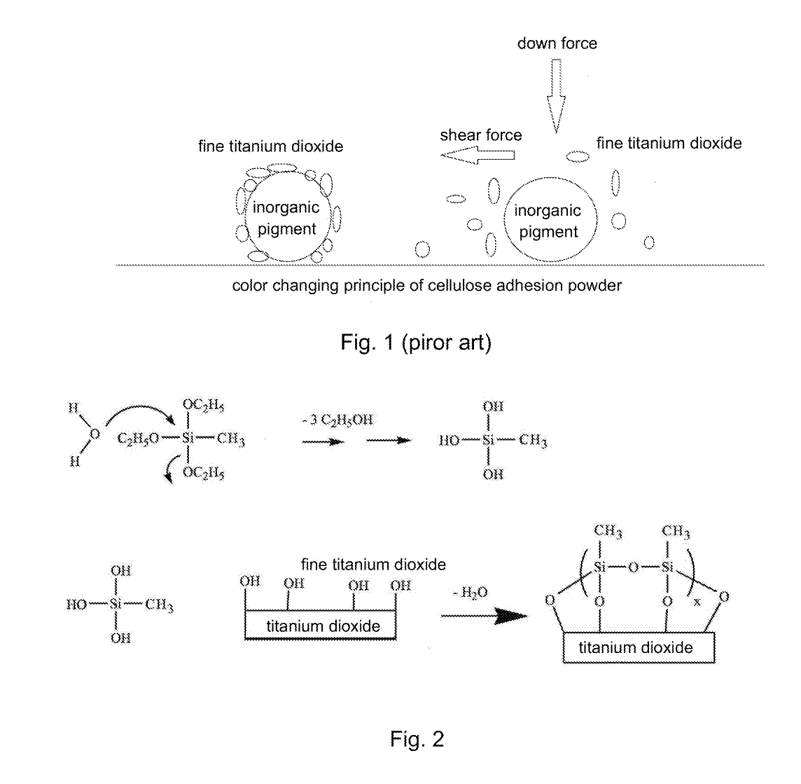

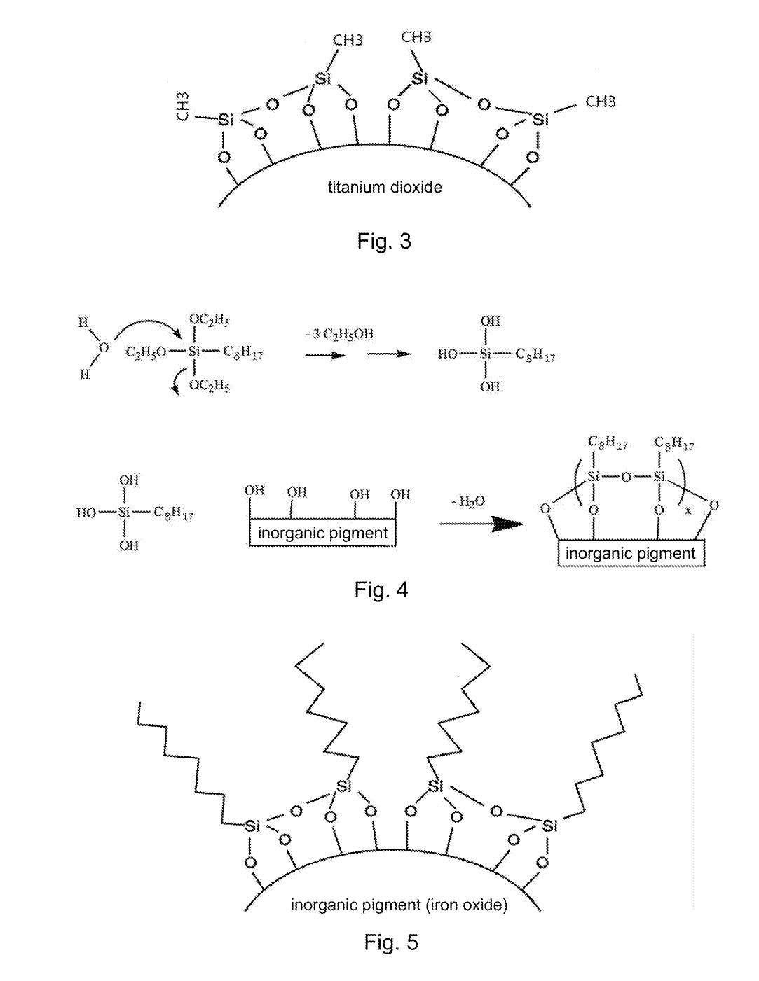

### DETAILED DESCRIPTION OF THE PREFERRED EMBODIMENT

Referring to preferred embodiments, the present invention is further illustrated.

The following descriptions are embodiments of colorant treatment methods:

A treatment method with “polydimethyl siloxane/vinyl polydimethyl siloxane polymer” is numbered as A1, a treatment method with “dimethiconol” is numbered as A2, a treatment method with “methyl triethoxy silane” is numbered as A3, a treatment method with “isooropyl titanium triisostearate” is numbered as B1, a treatment method with “stearic acid” is numbered as B2, and a treatment method with “octyl triethoxy silane” is numbered as B3.

### Embodiment 1

A treatment method is illustrated using a treatment (A1) of titanium dioxide with polydimethyl siloxane/vinyl polydimethyl siloxane polymer as follows:

dissolving 40 g polydimethyl siloxane/vinyl dimethicone polymer into 100 g cyclopentasiloxane, and thoroughly mixing for obtaining a surface treatment agent just before using;

adding 1 kg titanium dioxide into a high-speed mixer; under a high mixing speed, spraying the surface treatment agent prepared into the high-speed mixer with a spray tank; thoroughly stirring for thoroughly mixing the reaction solution with the titanium dioxide; and

discharging powder and instantly sending into an oven, heating at 105° C. for 4 h, and naturally cooling before discharging to obtain A1-titanium dioxide.

### Embodiment 2

A treatment method is illustrated using a treatment (B1) of iron oxide yellow with isooropyl titanium triisostearate as follows:

dissolving 40 g isooropyl titanium triisostearate into 120 g ethanol, and thoroughly mixing for obtaining a surface treatment agent just before using;

adding 1 kg iron oxide yellow into a high-speed mixer; under a high mixing speed, spraying the surface treatment agent prepared into the high-speed mixer with a spray tank; thoroughly stirring for thoroughly mixing the reaction solution with the iron oxide yellow; and

discharging powder and instantly sending into an oven, heating at 105° C. for 4 h, and naturally cooling before discharging to obtain B1-iron oxide yellow.

### Embodiment 3

A treatment method is illustrated using a treatment (B1) of iron oxide red with isooropyl titanium triisostearate as follows:

dissolving 40 g isooropyl titanium triisostearate into 120 g ethanol, and thoroughly mixing for obtaining a surface treatment agent just before using;

adding 1 kg iron oxide red into a high-speed mixer; under a high mixing speed, spraying the surface treatment agent prepared into the high-speed mixer with a spray tank; thoroughly stirring for thoroughly mixing the reaction solution with the iron oxide red; and

discharging powder and instantly sending into an oven, heating at 105° C. for 4 h, and naturally cooling before discharging to obtain B1-iron oxide red.

### Embodiment 4

A treatment method is illustrated using a treatment (B1) of iron oxide black with isooropyl titanium triisostearate as follows:

dissolving 40 g isooropyl titanium triisostearate into 120 g ethanol, and thoroughly mixing for obtaining a surface treatment agent just before using;

adding 1 kg iron oxide black into a high-speed mixer; under a high mixing speed, spraying the surface treatment agent prepared into the high-speed mixer with a spray tank; thoroughly stirring for thoroughly mixing the reaction solution with the iron oxide black; and

discharging powder and instantly sending into an oven, heating at 105° C. for 4 h, and naturally cooling before discharging to obtain B1-iron oxide black.

### Embodiment 5

A treatment method is illustrated using a treatment (A2) of iron oxide yellow with dimethiconol as follows:

dissolving 30 g dimethiconol into 90 g cyclopentasiloxane, and thoroughly mixing for obtaining a surface treatment agent just before using;

adding 1 kg iron oxide yellow into a high-speed mixer; under a high mixing speed, spraying the surface treatment agent prepared into the high-speed mixer with a spray tank; thoroughly stirring for thoroughly mixing the reaction solution with the iron oxide yellow; and

discharging powder and instantly sending into an oven, heating at 105° C. for 4 h, and naturally cooling before discharging to obtain A2-iron oxide yellow.

### Embodiment 6

A treatment method is illustrated using a treatment (A2) of iron oxide red with dimethiconol as follows:

replacing the iron oxide yellow in embodiment 5 with iron oxide red, and treating the iron oxide red by the method of embodiment 5 to obtain A2-iron oxide red.

### Embodiment 7

A treatment method is illustrated using a treatment (A2) of iron oxide black with dimethiconol as follows:

replacing the iron oxide yellow in embodiment 5 with iron oxide black, and treating the iron oxide black by the method of embodiment 5 to obtain A2-iron oxide black.

### Embodiment 8

A treatment method is illustrated using a treatment (B2) of titanium dioxide with stearic acid as follows:

dissolving 40 g stearic acid into 120 g ethanol, and thoroughly mixing for obtaining a surface treatment agent just before using;

adding 1 kg titanium dioxide into a high-speed mixer; under a high mixing speed, spraying the surface treatment agent prepared into the high-speed mixer with a spray tank; thoroughly stirring for thoroughly mixing the reaction solution with the titanium dioxide; and

discharging powder and instantly sending into an oven, heating at 105° C. for 4 h, and naturally cooling before discharging to obtain B2-titanium dioxide.

The colorants prepared by different surface treatments according to the above embodiments were applied to a specific foundation cream/liquid formula.

### Embodiment 9

The titanium oxide in embodiment 1 was replaced with iron oxide yellow, which was treated according to the technique of the embodiment 1 to obtain A1-iron oxide yellow.

### Embodiment 10

The titanium oxide in embodiment 1 was replaced with iron oxide red, which was treated according to the technique of embodiment 1 to obtain A1-iron oxide red.

### Embodiment 11

The titanium oxide in embodiment 1 was replaced with iron oxide yellow, which was treated according to the technique of embodiment 1 to obtain A1-iron oxide black.

### Embodiment 12

The iron oxide yellow in embodiment 2 was replaced with titanium oxide, which was treated according to the technique of embodiment 2 to obtain B1-titanium dioxide.

The following descriptions are embodiments of a preparation method of foundation cream/liquid with controllable color change.

### Embodiment 13

The colorants prepared in embodiments 1, 2, 3, and 4 above were added to a foundation cream formula in proportion, and made into samples according to the formula-specific preparation method of Table 2.

TABLE 2

| phase | | |
|---|---|---|
| number | component name | content (Wt) % |
| A | cetyl PEG/PPG-10/1 polydimethyl siloxane | 1.5 |
| (oil phase) | PEG-10 polydimethyl siloxane | 0.75 |
| | PEG/PPG-18/18 polydimethyl siloxane, | 2.00 |
| | cyclopentasiloxane | |
| | polydimethyl siloxane cross-linked polymer, | 1.00 |
| | cyclopentasiloxane | |
| | cyclopentasiloxane | 5.00 |
| | C9-12 alkane, coco-caprylate/caprate | 4.00 |
| | isononyl isononanoate | 2.10 |
| | disteardimonium hectorite | 0.50 |
| | phenyl trimethicone | 1.60 |
| | polydimethyl siloxane | 3.00 |
| B | A1-titanium dioxide | 8.00 |
| | B1-iron oxide yellow | 1.30 |
| | B1-iron oxide red | 0.83 |
| | B1-iron oxide black | 0.37 |
| C | deionized water | 100 |
| (water | 1% ethyloic-β-glucan | 3.00 |
| phase) | sodium chloride | 1.00 |
| | betaine | 3.00 |
| | glycerin | 5.00 |
| | 1,3-butanediol | 10.00 |
| D | preservative | appropriate |
| | | amount |
| | fragrance | appropriate |
| | | amount |

A preparation method comprises steps of:

(1) stirring an A phase at room temperature for 5 min for thoroughly mixing raw materials;

(2) heating the A phase to 55-60° C., then adding B phase powder, and keeping homogeneity for 1 min for thoroughly dispersing the powder;

(3) meanwhile, heating a C phase to about 55-60° C. for completely melting; (4) increasing a stirring speed of the A and B phases (to 200-300 rpm), slowly adding the C phase into the A and B phases for completely emulsifying; and

(5) cooling by stirring until a temperature is lower than 45° C., then adding a D phase in such a manner that cream becomes white, and discharging.

A white foundation cream a is finally obtained, which becomes a light skin color when applied on the skin.

### Embodiment 14

The colorants prepared in embodiments 5, 6, 7, and 8 above were added to a foundation cream formula in proportion, and made into samples according to the formula-specific preparation method of Table 3.

TABLE 3

| phase | | |
|---|---|---|
| number | component name | content (Wt) % |
| A | cetyl PEG/PPG-10/1 polydimethyl siloxane | 1.5 |
| (oil phase) | PEG-10 polydimethyl siloxane | 0.75 |
| | PEG/PPG-18/18 polydimethyl siloxane, | 2.00 |
| | cyclopentasiloxane | |
| | polydimethyl siloxane cross-linked polymer, | 1.00 |
| | cyclopentasiloxane | |
| | cyclopentasiloxane | 5.00 |
| | C9-12 alkane, coco-caprylate/caprate | 4.00 |
| | isononyl isononanoate | 2.10 |
| | disteardimonium hectorite | 0.50 |
| | phenyl trimethicone | 1.60 |
| | polydimethyl siloxane | 3.00 |
| B | B2-titanium dioxide | 9.00 |
| | A2-iron oxide yellow | 1.50 |
| | A2-iron oxide red | 0.80 |
| | A2-iron oxide black | 0.33 |
| C | deionized water | 100 |
| (water | 1% ethyloic-β-glucan | 3.00 |
| phase) | sodium chloride | 1.00 |
| | betaine | 3.00 |
| | glycerin | 5.00 |
| | 1,3-butanediol | 10.00 |
| D | preservative | appropriate |
| | | amount |
| | fragrance | appropriate |
| | | amount |

A preparation method comprises steps of:

(1) stirring an A phase at room temperature for 5 min for thoroughly mixing raw materials;

(2) heating the A phase to 55-60° C., then adding B phase powder, and keeping homogeneity for 1 min for thoroughly dispersing the powder;

(3) meanwhile, heating a C phase to about 55-60° C. for completely melting;

(4) increasing a stirring speed of the A and B phases (to 200-300 rpm), slowly adding the C phase into the A and B phases for completely emulsifying; and

(5) cooling by stirring until a temperature is lower than 45° C., then adding a D phase in such a manner that cream has a stable color (a dark skin color, which does not change by continuously stirring 10 min), and discharging.

A foundation cream b with a dark skin color is finally obtained, which becomes a light skin color when applied on the skin.

In order to highlight the beneficial effects of the foundation cream/foundation liquid with a controllable color change according to the present invention, the following comparative experiments were also performed.

### Comparative Example 1

The colorants prepared in embodiments 1, 9, 10 and 11 above were added to a foundation cream formula of Table 4 in proportion, and made into samples according to the method of embodiment 13.

TABLE 4

| phase | | |
|---|---|---|
| number | component name | content (Wt) % |
| A | cetyl PEG/PPG-10/1 polydimethyl siloxane | 1.5 |
| (oil phase) | PEG-10 polydimethyl siloxane | 0.75 |
| | PEG/PPG-18/18 polydimethyl siloxane, | 2.00 |
| | cyclopentasiloxane | |
| | polydimethyl siloxane cross-linked polymer, | 1.00 |
| | cyclopentasiloxane | |
| | cyclopentasiloxane | 5.00 |
| | C9-12 alkane, coco-caprylate/caprate | 4.00 |
| | isononyl isononanoate | 2.10 |
| | disteardimonium hectorite | 0.50 |
| | phenyl trimethicone | 1.60 |
| | polydimethyl siloxane | 3.00 |
| B | A1-titanium dioxide | 8.00 |
| | A1-iron oxide yellow | 1.30 |
| | A1-iron oxide red | 0.83 |
| | A1-iron oxide black | 0.37 |
| C | deionized water | 100 |
| (water | 1% ethyloic-β-glucan | 3.00 |
| phase) | sodium chloride | 1.00 |
| | betaine | 3.00 |
| | glycerin | 5.00 |
| | 1,3-butanediol | 10.00 |
| D | preservative | appropriate |
| | | amount |
| | fragrance | appropriate |
| | | amount |

During the preparation process, it is found that after the addition of all the materials, the color of the foundation cream is always a skin color, the appearance of the cream does not turn white, the color does not change when the cream is applied on the skin, and this foundation cream is numbered as foundation cream c.

### Comparative Example 2

The colorants prepared in embodiments 12, 2, 3 and 4 above were added to a foundation cream formula of Table 5 in proportion, and made into samples according to the method of embodiment 13.

TABLE 5

| phase | | |
|---|---|---|
| number | component name | content (Wt) % |
| A | cetyl PEG/PPG-10/1 polydimethyl siloxane | 1.5 |
| (oil phase) | PEG-10 polydimethyl siloxane | 0.75 |
| | PEG/PPG-18/18 polydimethyl siloxane, | 2.00 |
| | cyclopentasiloxane | |
| | polydimethyl siloxane cross-linked polymer, | 1.00 |
| | cyclopentasiloxane | |
| | cyclopentasiloxane | 5.00 |
| | C9-12 alkane, coco-caprylate/caprate | 4.00 |
| | isononyl isononanoate | 2.10 |
| | disteardimonium hectorite | 0.50 |
| | phenyl trimethicone | 1.60 |
| | polydimethyl siloxane | 3.00 |
| B | B1-titanium dioxide | 8.00 |
| | B1-iron oxide yellow | 1.30 |
| | B1-iron oxide red | 0.83 |
| | B1-iron oxide black | 0.37 |
| C | deionized water | 100 |
| (water | 1% ethyloic-β-glucan | 3.00 |
| phase) | sodium chloride | 1.00 |
| | betaine | 3.00 |
| | glycerin | 5.00 |
| | 1,3-butanediol | 10.00 |
| D | preservative | appropriate |
| | | amount |
| | fragrance | appropriate |
| | | amount |

During the preparation process, it is found that after the addition of all the materials, the color of the foundation cream is always a skin color and does not change when the cream is applied on the skin, and this foundation cream is numbered as foundation cream d.

If the white colorant and the chromatic colorant are not directly added to the foundation formula, but are pre-mixed into pre-mixed powder, the pre-mixed powder is then added to the foundation formula, as illustrated by the following embodiments.

### Embodiment 15

A method for treating titanium dioxide in a white colorant by methyl triethoxy silane (A3) comprises steps of:

thoroughly mixing 0.45 kg methyl triethoxy silane with 1.05 kg 95% ethanol for obtaining a surface treatment agent just before using;

adding 15 kg titanium dioxide into a high-speed mixer; under a high mixing speed, spraying the surface treatment agent prepared into the high-speed mixer with a spray tank, and thoroughly stirring for thoroughly mixing the surface treatment agent with the titanium dioxide; and

discharging powder and instantly sending into an oven, heating at 105° C. for 4 h, and naturally cooling before discharging to obtain A3-titanium dioxide.

### Embodiment 16

A method for treating iron oxide yellow in a chromatic colorant by octyl triethoxy silane (B3) comprises steps of:

thoroughly mixing 0.45 kg octyl triethoxy silane with 1.05 kg 95% ethanol for obtaining a surface treatment agent just before using;

adding 15 kg iron oxide yellow inorganic pigment into a high-speed mixer; under a high mixing speed, spraying the surface treatment agent prepared into the high-speed mixer with a spray tank, and thoroughly stirring for thoroughly mixing the surface treatment agent with the inorganic pigment; and

discharging powder and instantly sending into an oven, heating at 105° C., or 70° C. if the inorganic pigment is the iron oxide yellow, for 4 h, and naturally cooling before discharging to obtain B3-iron oxide yellow.

### Embodiment 17

A method for treating iron oxide red in a chromatic colorant by octyl triethoxy silane (B3) is as follows:

replacing the iron oxide yellow in embodiment 16 with iron oxide red, and treating the iron oxide red by the method of embodiment 16 to obtain B3-iron oxide red.

### Embodiment 18

A method for treating iron oxide black in a chromatic colorant by octyl triethoxy silane (B3) is as follows:

replacing the iron oxide yellow in embodiment 16 with iron oxide black, and treating the iron oxide black by the method of embodiment 16 to obtain B3-iron oxide black.

### Embodiment 19

color-changeable pre-mixed powder with different proportions, wherein following proportions are all based on mass fraction.

TABLE 6

| | CC803 | CC805 | CC807 | CC808 |
|---|---|---|---|---|
| | content | content | content | content |
| component name | (%) | (%) | (%) | (%) |
| A3-titanium dioxide (the | 88 | 90 | 85 | 91 |
| titanium dioxide treated by | | | | |
| methyl triethoxy silane) | | | | |
| B3-iron oxide yellow (the | 8.7 | 8 | 9.6 | 5.4 |
| iron oxide yellow treated by | | | | |
| octyl triethoxy silane) | | | | |
| B3-iron oxide red (the iron | 2.3 | 1.4 | 4 | 2.3 |
| oxide red treated by octyl | | | | |
| triethoxy silane) | | | | |
| B3-iron oxide black (the iron | 1 | 0.6 | 1.4 | 1.3 |
| oxide black treated by octyl | | | | |
| triethoxy silane) | | | | |

Embodiment 20: color-changeable foundation cream

The formula is:

TABLE 7

| phase | | content (Wt) % | | |
|---|---|---|---|---|
| number | component name/provider | formula 1 | formula 2 | formula 3 |
| A | Abil EM180 (Degussa) | 0.75 | 0.75 | 0.70 |
| | Abil EM90 (Degussa) | 0.75 | 0.75 | 0.70 |
| | Sensil 5060 silane emollient | 2.20 | 2.20 | 2.20 |
| | (Shanghai Scinecoo) | | | |
| | DC9040 silicone elastomer | 1.00 | 1.00 | 1.00 |
| | (Dow Corning) | | | |
| | DC345 (Dow Corning) | 5.00 | 5.00 | 6.00 |
| | DC556 (Dow Corning) | 1.60 | 1.60 | 1.60 |
| | DC200 (5CST) | 5.00 | 5.00 | 6.00 |
| | (Dow Corning) | | | |
| B | color-changeable pre-mixed | 8.00 | 10.0 | 11.0 |
| | powder | | | |
| | CC807/CC803/CC805/CC808 | | | |
| C | deionized water | 100 | 100 | 100 |
| | 1% ethyloic-β-glucan (Angel | 3.00 | 3.00 | 3.00 |
| | Yeast C90) | | | |
| | sodium chloride | 1.00 | 1.00 | 1.00 |
| | betaine(trimethylglycine) | 3.00 | 3.00 | 3.00 |
| | 1,3-butanediol | 15.00 | 15.00 | 15.00 |
| D | preservative | appropriate | appropriate | appropriate |
| | | amount | amount | amount |
| | fragrance | appropriate | appropriate | appropriate |
| | | amount | amount | amount |

The preparation method comprises steps of:

1) mixing and stirring an A phase, keeping homogeneity for 1 min, and thoroughly dispersing with a DC9040 silicone elastomer;

2) heating the A phase to 55-60° C., then adding B phase powder, and keeping homogeneity for 1 min for thoroughly dispersing the powder;

3) meanwhile, heating a C phase to about 55-60° C. for completely melting;

4) increasing a stirring speed of the A and B phases (to 200-300 rpm), slowly adding the C phase into the A and B phases for completely emulsifying;

5) cooling by stirring until a temperature is lower than 45° C., then adding a D phase in such a manner that cream becomes white, and discharging; and

6) during cooling, keeping a stirring speed (at 200-300 rpm), wherein the cream becomes white within 2-3 h.

White foundation creams are finally obtained, which becomes a light skin color when applied on the skin.

In order to further illustrate the influence of different ratios of the white colorant to the chromatic colorant on the color changing effect of the foundation, the following embodiments are added.

### Embodiment 21

The colorants prepared in embodiments 1, 2, 3, and 4 above were added to a foundation cream formula in proportion, and made into samples according to the formula-specific preparation method of the following table.

TABLE 8

| | | content (Wt) % | | |
|---|---|---|---|---|
| phase | | foundation | foundation | foundation |
| number | component name | cream a-1 | cream a-2 | cream a-3 |
| A | cetyl PEG/PPG-10/1 polydimethyl siloxane | 1.5 | 1.5 | 1.5 |
| (oil | PEG-10 polydimethyl siloxane | 0.75 | 0.75 | 0.75 |
| phase) | PEG/PPG-18/18 polydimethyl siloxane, | 2.00 | 2.00 | 2.00 |
| | cyclopentasiloxane | | | |
| | polydimethyl siloxane cross-linked polymer, | 1.00 | 1.00 | 1.00 |
| | cyclopentasiloxane | | | |
| | cyclopentasiloxane | 5.00 | 5.00 | 5.00 |
| | C9-12 alkane, coco-caprylate/caprate | 4.00 | 4.00 | 4.00 |
| | isononyl isononanoate | 2.10 | 2.10 | 2.10 |
| | disteardimonium hectorite | 0.50 | 0.50 | 0.50 |
| | phenyl trimethicone | 1.60 | 1.60 | 1.60 |
| | polydimethyl siloxane | 3.00 | 3.00 | 3.00 |
| B | A1-titanium dioxide | 10.68 | 10.92 | 11.16 |
| | B1-iron oxide yellow | 0.79 | 0.67 | 0.57 |
| | B1-iron oxide red | 0.32 | 0.24 | 0.16 |
| | B1-iron oxide black | 0.21 | 0.17 | 0.11 |
| C | deionized water | 100 | 100 | 100 |
| (water | 1% ethyloic-β-glucan | 3.00 | 3.00 | 3.00 |
| phase) | sodium chloride | 1.00 | 1.00 | 1.00 |
| | betaine | 3.00 | 3.00 | 3.00 |
| | glycerin | 5.00 | 5.00 | 5.00 |
| | 1,3-butanediol | 10.00 | 10.00 | 10.00 |
| D | preservative | appropriate | appropriate | appropriate |
| | | amount | amount | amount |
| | fragrance | appropriate | appropriate | appropriate |
| | | amount | amount | amount |

A preparation method comprises steps of:

(1) stirring an A phase at room temperature for 5 min for thoroughly mixing raw materials;

(2) heating the A phase to 55-60° C., then adding B phase powder, and keeping homogeneity for 1 min for thoroughly dispersing the powder;

(3) meanwhile, heating a C phase to about 55-60° C. for completely melting;

(4) increasing a stirring speed of the A and B phases (to 200-300 rpm), slowly adding the C phase into the A and B phases for completely emulsifying; and

(5) cooling by stirring until a temperature is lower than 45° C., then adding a D phase in such a manner that cream becomes white, and discharging.

The foundations prepared according to the addition ratios of three different white colorants and a chromatic colorant are numbered as foundation cream a-1, foundation cream a-2 and foundation cream a-3, which are all white. When applied on the skin, colors of the foundation cream a-1, the foundation cream a-2 and the foundation cream a-3 become dark, but the colors of the foundation cream a-1 and the foundation cream a-2 change obviously, whereas the color of the foundation cream a-3 changes little, in which the foundation formula contains little chromatic colorant, and the color represented finally is very light after applied to the skin, so the visual contrast formed is small.

### Embodiment 22

The colorants prepared in embodiments 9, 10, 11 and 12 above were added to a foundation cream formula in proportion, and made into samples according to the formula-specific preparation method of the following table.

TABLE 9

| | | content (Wt) % | | |
|---|---|---|---|---|
| phase | | foundation | foundation | foundation |
| number | component name | cream b-1 | cream b-2 | cream b-3 |
| A | cetyl PEG/PPG-10/1 polydimethyl siloxane | 1.5 | 1.5 | 1.5 |
| (oil | PEG-10 polydimethyl siloxane | 0.75 | 0.75 | 0.75 |
| phase) | PEG/PPG-18/18 polydimethyl siloxane, | 2.00 | 2.00 | 2.00 |
| | cyclopentasiloxane | | | |
| | polydimethyl siloxane cross-linked polymer, | 1.00 | 1.00 | 1.00 |
| | cyclopentasiloxane | | | |
| | cyclopentasiloxane | 5.00 | 5.00 | 5.00 |
| | C9-12 alkane, coco-caprylate/caprate | 4.00 | 4.00 | 4.00 |
| | isononyl isononanoate | 2.10 | 2.10 | 2.10 |
| | disteardimonium hectorite | 0.50 | 0.50 | 0.50 |
| | phenyl trimethicone | 1.60 | 1.60 | 1.60 |
| | polydimethyl siloxane | 3.00 | 3.00 | 3.00 |
| B | B1-titanium dioxide | 10.20 | 10.68 | 11.16 |
| | A1-iron oxide yellow | 1.02 | 0.79 | 0.57 |
| | A1-iron oxide red | 0.48 | 0.32 | 0.16 |
| | A1-iron oxide black | 0.30 | 0.21 | 0.11 |
| C | deionized water | 100 | 100 | 100 |
| (water | 1% ethyloic-β-glucan | 3.00 | 3.00 | 3.00 |
| phase) | sodium chloride | 1.00 | 1.00 | 1.00 |
| | betaine | 3.00 | 3.00 | 3.00 |
| | glycerin | 5.00 | 5.00 | 5.00 |
| | 1,3-butanediol | 10.00 | 10.00 | 10.00 |
| D | preservative | appropriate | appropriate | appropriate |
| | | amount | amount | amount |
| | fragrance | appropriate | appropriate | appropriate |
| | | amount | amount | amount |

A preparation method comprises steps of:

(1) stirring an A phase at room temperature for 5 min for thoroughly mixing raw materials;

(2) heating the A phase to 55-60° C., then adding B phase powder, and keeping homogeneity for 1 min for thoroughly dispersing the powder;

(3) meanwhile, heating a C phase to about 55-60° C. for completely melting;

(4) increasing a stirring speed of the A and B phases (to 200-300 rpm), slowly adding the C phase into the A and B phases for completely emulsifying; and

(5) cooling by stirring until a temperature is lower than 45° C., then adding a D phase in such a manner that cream has a stable color (a dark skin color, which does not change by continuously stirring 10 min), and discharging.

The foundations prepared according to the addition ratios of three different white colorants and a chromatic colorant are numbered as foundation cream b-1, foundation cream b-2 and foundation cream b-3, which have different colors, wherein the foundation cream b-1 has a honey color, the foundation cream b-2 has an ivory color, and the foundation cream b-3 has a light skin color. When applied on the skin, colors of the foundation cream b-1, the foundation cream b-2 and the foundation cream b-3 all become dark, but the colors of the foundation cream b-1 and the foundation cream b-2 change obviously, whereas the color of the foundation cream b-3 changes little, in which the foundation formula contains little chromatic colorant, and the color represented finally is very light after applied to the skin, so the visual contrast formed is small.

The following three stability tests were carried out on the color-changeable foundation creams prepared in embodiments 13, 14, 21 and 22, under 3 months of 48° C. heat storage, three times of −10 to 48° C. freeze-thaw cycles and preservation at normal temperature for one year.

The stability test results are as follows:

TABLE 10

| | 3 months | three times | preservation at |
|---|---|---|---|
| foundation | of 48° C. heat | of −10 to 48° C. | normal temperature |
| cream No. | storage | freeze-thaw cycles | for one year |
| foundation | stable, | stable, no bleeding | stable, no bleeding |
| cream a | no bleeding | | |
| foundation | stable, | stable, no bleeding | stable, no bleeding |
| cream b | no bleeding | | |
| foundation | stable, | stable, no bleeding | stable, no bleeding |
| cream a-1 | no bleeding | | |
| foundation | stable, | stable, no bleeding | stable, no bleeding |
| cream a-2 | no bleeding | | |
| foundation | stable, | stable, no bleeding | stable, no bleeding |
| cream a-3 | no bleeding | | |
| foundation | stable, | stable, no bleeding | stable, no bleeding |
| cream b-1 | no bleeding | | |
| foundation | stable, | stable, no bleeding | stable, no bleeding |
| cream b-2 | no bleeding | | |
| foundation | stable, | stable, no bleeding | stable, no bleeding |
| cream b-3 | no bleeding | | |

It can be seen from the stability test data that the color-changeable foundation prepared by the present invention has a stable appearance in the storage process regardless of a color change from light to dark or from dark to light during smearing, and no “bleeding phenomenon” occurs.

Trial Survey:

After trials, it is found that the color-changeable foundation cream a-1 and the color-changeable foundation cream b-1 obtained according to embodiments 13 and 14 of the present invention are significant in the color changing effect, rapid in color change, strong in covering ability and easy to evenly smear. 30 testers were invited for trying the present invention (all females, 20-40 years old; testing time: February, 2018; testing positions: 10 testers in each of Shanghai, Shenzhen and Beijing). Questionnaires were filled after using. The trial results are summarized as follows:

TABLE 11

| effect | foundation cream a-1 | foundation cream b-1 |
|---|---|---|
| color change speed | 4.85 | 4.84 |
| strong covering | 4.62 | 4.37 |
| ability | | |
| soft and silky feeling | 4.90 | 4.45 |
| breathability | 4.63 | 4.50 |
| endurance | 4.37 | 4.75 |
| wettability | 4.40 | 4.28 |
| number of persons | 28 | 25 |
| with purchase | | |
| intention | | |

The scoring criteria are as follows: the color change speed scores 5, 4, 3, 2, 1 from fast to slow; the covering ability scores 5, 4, 3, 2, 1 from strong to weak; the soft and silky feeling scores 5, 4, 3, 2, 1 from high to low; the breathability scores 5, 4, 3, 2, 1 from strong to weak; the endurance scores 5, 4, 3, 2, 1 from strong to weak; and the wettability scores 5, 4, 3, 2, 1 from strong to weak. The number of persons with purchase intention is the number of the persons who are willing to buy when commercially available.

One skilled in the art will understand that the preferred embodiments of the present invention as described above are exemplary only and not intended to be limiting. The preferred embodiments are subject to change without departure from such principles. Therefore, this invention includes all modifications encompassed within the spirit and scope of the following claims.

### INDUSTRIAL PRACTICABILITY

According to the present invention, the formula of the foundation with the controllable color change and the preparation method thereof adopt a certain formula and an optimized method, wherein the foundation comprises a white colorant and a chromatic colorant; the white colorant and the chromatic colorant are treated by different surface treatment methods. During use, the color can change rapidly, and the effect is optimal. According to the present invention, the foundation with the controllable color change has a rapid color changing effect, a strong covering ability, varied colors, adjustable chroma, safety, and simple production processes, which enables a great market prospect and a strong industrial practicability.

1. A foundation with a controllable color change, comprising colorants, wherein the colorants comprise a white colorant and a chromatic colorant; the white colorant and the chromatic colorant are treated by different surface treatment methods; the surface treatment methods comprise a silicone-philic treatment method and an ester-philic silicone-phobic treatment method; and the foundation changes from an original light color to a dark color or from an original dark color to a light color after smearing.

2. The foundation, as recited in claim 1, wherein when the foundation changes from an original light color to a dark color after smearing, the silicone-philic treatment is adopted for the white colorant in the foundation, and the ester-philic silicone-phobic treatment is adopted for the chromatic colorant in the foundation;
when the foundation changes from an original dark color to a light color after smearing, the ester-philic silicone-phobic treatment is adopted for the white colorant in the foundation, and the silicone-philic treatment is adopted for the chromatic colorant in the foundation.

3. The foundation, as recited in claim 2, wherein the total amount of the colorants is 1 to 25%; the amount of the white colorant is 70 to 98% of the total amount of the colorants, and the rest is the chromatic colorant.

4. The foundation, as recited in claim 3, wherein when the foundation changes from an original light color to a dark color after smearing, the total amount of the colorants in the foundation is 2 to 25%, preferably 5 to 20%, more preferably 8 to 15%, based on the total mass of the foundation;
the adding concentration of a surface treatment agent adopted by the white colorant is 0.5% to 12%, preferably 1.5% to 9%, more preferably 2% to 5%, based on the mass of the white colorant; the amount of the white colorant in the foundation is 83 to 98%, preferably 85 to 95%, more preferably 87 to 92%, based on the total amount of the colorants;
the adding concentration of a surface treatment agent adopted by the chromatic colorant is 0.5% to 12%, preferably 1.5% to 9%, more preferably 2% to 6%, based on the mass of the chromatic colorant; the amount of the chromatic colorant in the foundation is 2 to 17%, preferably 5 to 15%, more preferably 8 to 13%, based on the total amount of the colorants.

5. The foundation, as recited in claim 3, wherein when the foundation changes from an original dark color to a light color after smearing, the total amount of the colorants in the foundation is 2 to 25%, preferably 5 to 20%, more preferably 6 to 14%, based on the total mass of the foundation;
the adding concentration of a surface treatment agent adopted by the white colorant is 0.5% to 11%, preferably 1% to 8%, more preferably 2% to 5%, based on the mass of the white colorant; the amount of the white colorant in the foundation is 73 to 95%, preferably 78 to 92%, more preferably 82 to 90%, based on the total amount of the colorants;
the adding concentration of a surface treatment agent adopted by the chromatic colorant is 0.5% to 12%, preferably 1.5% to 8%, more preferably 2% to 6%, based on the mass of the chromatic colorant; the amount of the chromatic colorant in the foundation is 5 to 27%, preferably 8 to 22%, more preferably 10 to 18%, based on the total amount of the colorants.

6. The foundation, as recited in claim 3, wherein the white colorant is one or more of titanium dioxide, zinc oxide and barium sulfate; and the chromatic colorant is one or more of iron oxide yellow, iron oxide red, iron oxide black, chromium oxide green, chromium hydroxide green, ultramarine blue, ultramarine purple and ultramarine pink.

7. The foundation, as recited in claim 4, wherein the foundation is of a water-in-oil type, the oil phase is a continuous phase, and the water phase is a dispersed phase.

8. The foundation, as recited in claim 5, wherein the oil phase comprises a water-in-oil emulsifier, and the water-in-oil emulsifier may be selected from one or more of PEG-10 polydimethyl siloxane, PEG-12 polydimethyl siloxane, PEG-9 polydimethyl siloxyethyl dimethicone, lauryl PEG-9 polydimethyl siloxyethyl dimethicone, cetyl PEG/PPG-10/1 polydimethyl siloxane, lauryl PEG/PPG-18/18 polydimethyl siloxane, PEG-12 polymethyl siloxane/PPG-20 cross-linked polymer and PEG/PPG-18/18 polydimethyl siloxane, added in an amount of 0.1 to 10%.

9. The foundation, as recited in claim 5, wherein the oil phase comprises an emollient, which may be selected from silicone oil, natural oil or synthetic ester;
the silicone oil may be selected from one or more of polydimethyl siloxane, cyclopentasiloxane, cyclohexasiloxane, methyl polytrimethyl siloxane, octyl polymethyl siloxane and phenyl polytrimethylsiloxane, added in an amount of 1 to 50%;
the natural oil may be selected from one or more of coco-caprylate/caprate, C9-12 alkane, castor oil, hydrogenated castor oil, jojoba oil and caprylic/capric triglyceride, added in an amount of 0.1˜10%;
the synthetic ester may be selected from one or more of propyl heptyl caprylate, dioctyl carbonate, caprylic/capric/myristic/stearic triglyceride, isopropyl myristate, isononyl isononanoate, hydrogenated polydecene, octyldodecanol myristate and hydrogenated polyisobutylene, added in an amount of 0.1 to 10%.

10. The foundation, as recited in claim 5, wherein the oil phase comprises a thickening suspension, which may be selected from one or more of disteardimonium hectorite, quaternary ammonium salt-18 bentonite and stearalkonium hectorite, added in an amount of 0.1 to 5%.

11. The foundation, as recited in claim 5, wherein the water phase comprises deionized water, added in an amount of 10 to 60%.

12. The foundation, as recited in claim 5, wherein the water phase comprises a humectant, which may be selected from at least two of glycerin, propylene glycol, butanediol, pentanediol and hexanediol, added in an amount of 1 to 30%.

13. The foundation, as recited in claim 1, wherein the foundation is foundation cream or foundation liquid.

14. A preparation method, as recited in claim 1, comprising the following steps of:
(1) colorant treatment: if a silicone-philic treatment is adopted for the white colorant, adopting an ester-philic silicone-phobic treatment for the chromatic colorant; if an ester-philic silicone-phobic treatment is adopted for the white colorant, adopting a silicone-philic treatment for the chromatic colorant;
(2) stirring oil phase components in the foundation formula at room temperature for thoroughly mixing the raw materials;
(3) heating the oil phase mixed thoroughly to 55-60° C., then adding the colorants treated in step (1), and keeping homogeneity for 1 min for thoroughly dispersing the powder;
(4) heating the water phase component in the foundation formula to about 55-60° C. for completely melting;
(5) controlling a stirring speed of the materials mixed thoroughly in step (3) to 200-300 rpm, and slowly adding the water phase component thereto for completely emulsifying; and
(6) cooling by stirring until a temperature is lower than 45° C., adding a preservative and an fragrance, and continuously stirring until the color of the product is stable.
